(19) United States
(12) Patent Application Publication
(10) Pub. No.: US 2021/0401526 A1
(43) Pub. Date: Dec. 30, 2021

(54) PROPELLING DEVICES FOR PROPELLING THROUGH A MEDIUM, USING EXTERNAL MAGNETIC STIMULI APPLIED THEREON

(71) Applicants: Michael SHPIGELMACHER, Los Angeles, CA (US); Alex KISELYOV, San Diego, CA (US); Hovhannes SARGSYAN, North Hollywood, CA (US); John CAPUTO, Los Angeles, CA (US); Eli VAN CLEVE, Los Angeles, CA (US); Eran OREN, Tel Aviv (IL); Suchyun CHO, Los Angeles, CA (US); BIONAUT LABS LTD., Herzliya (IL)

(72) Inventors: Michael SHPIGELMACHER, Los Angeles, CA (US); Alex KISELYOV, San Diego, CA (US); Hovhannes SARGSYAN, North Hollywood, CA (US); John CAPUTO, Los Angeles, CA (US); Eli VAN CLEVE, Los Angeles, CA (US); Eran OREN, Tel Aviv (IL); Suehyun CHO, Los Angeles, CA (US)

(73) Assignee: BIONAUT LABS LTD., Herzliya (IL)

(21) Appl. No.: 17/289,675

(22) PCT Filed: Oct. 31, 2019
(86) PCT No.: PCT/US2019/059178

§ 371 Date: Apr. 28, 2021

Related U.S. Application Data
(60) Provisional application No. 62/754,948, filed on Nov. 2, 2018.

(51) Int. Cl. A61B 34/00; A61K 49/00

(52) U.S. Cl. CPC A61B 34/73; A61B 2034/302; A61K 49/0002

(57) ABSTRACT

A propelling device and methods of use thereof. The device is configured to propel through a medium, using external magnetic stimuli applied thereon; the device comprises: a propelling-element and a magnet in communication with the propelling element. The magnet is configured to respond to the applied magnetic stimuli and to rotate the propelling-element; the propelling-element is configured to convert rotary motion thereof into translation motion, and thereby to propel the device through the medium.

### BACKGROUND OF THE INVENTION

Reproducible and accurate propulsion of nano-/micro-particles in different biological matrices poses a formidable challenge. Controlled motion of a micro robot (also noted as “microbot”) in a biologically or medically relevant environment depends on reliable external force, as well as on the properties of respective nano-/micro-particles of the microbot.

Both normal and pathological tissues exhibit distinct biophysical microscale features, posing specific requirements on the particle's Shape-, Size-, Surface- and material-properties (e.g. Stiffness)—also noted as “4S properties.” Accordingly, there is a need for microbots that would answer these 4S properties challenges.

### SUMMARY OF THE INVENTION

According to some embodiments of the invention, a propelling device and methods of use thereof are provided; the device is configured to propel through a medium, using external magnetic stimuli applied thereon; the device comprising: a propelling-element and a magnet in communication with the propelling element. According to some embodiments, the magnet is configured to respond to the applied magnetic stimuli and to rotate the propelling-element; the propelling-element is configured to convert rotary motion thereof into translation motion, and thereby to propel the device through the medium.

According to some embodiments of the invention, a propelling device is provided, configured to propel through a medium, using external magnetic stimuli applied thereon, the device comprising:
a helical spring-like element; and
a cube, cuboid, prism, ellipsoid, disc-like, cylindrical magnet, accommodated within the helical element, wherein their longitudinal axes are aligned.

According to some embodiments, the magnet is configured to respond to the applied magnetic stimuli and to rotate the helical element; and wherein the helical element is configured to convert rotary motion thereof into a translation motion along at least one of: the longitudinal axis, 2D trajectory, 3D trajectory; and thereby to propel the device through the medium.

According to some embodiments one of the following holds true:
the medium comprises at least one material selected from: viscoelastic medium, extracellular matrix, interstitial space, biological compartment, biological duct, biological vessel, biological node, biological tissue, biological organ;
the helical element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
   at least one nickel-plated neodymium optionally selected from: N35, N38, N40, N42, N45, N48, N50, N52, and N55; or
   at least one alternative permanent nano/micro magnet material selected from: samarium cobalt (SmCo), alnico, ceramic, ferrite.

According to some embodiments, the front end of the helical element comprises a sharp and/or chiseled tip.

According to some embodiments, the magnet is accommodated at a front section, at a center section, or at a back section of the helical element.

According to some embodiments, the magnet is encased with a layer of titanium vessel.

According to some embodiments, at least part of the device is covered with—or embedded into a matrix that contains—an imaging agent, configured to facilitate visualization; the imaging agent optionally comprising at least one of: Rhodamine B, Fluorescein, microbubbles, microdefects, mesoporous silica nano- and micro-particles, and Upconversion Phosphors.

According to some embodiments, the magnet is fixed to the helical element, optionally via an adhesive material comprising at least one of: epoxy, acrylics, polyurethane, UV curable, and cyanoacrylate based materials.

According to some embodiments, the adhesive material is incorporated with mesoporous nano- or micro-silica particles, configured to enhance contrast under ultrasound radiation.

According to some embodiments:
the helical element comprises:
   outer diameter ranging between 0.66-1.2 mm;
   inner diameter ranging between 0.3-1.1 mm;
   pitch length ranging between 0.5-2.2 mm;
   length ranging between 1-5.6 mm;
the magnet comprises:
   diameter ranging between 0.3-0.8 mm;
   length ranging between 0.5-1.5 mm.

According to some embodiments od the invention, a propelling device is provided, configured to propel through a medium, using external magnetic stimuli applied thereon, the device comprising:
a screw-like element characterized by conical- or cylindrical-core and a helical ridge;
a cylindrical magnet, accommodated within a hole drilled in the cylindrical core, wherein their longitudinal axes are aligned.

According to some embodiments, the magnet is accommodated at a front section or a back section of the cylindrical core.

According to some embodiments, magnet is configured to respond to the applied magnetic stimuli and to rotate the helical element; and wherein the screw-like element is configured to convert rotary motion thereof into translation motion along at least one of: the longitudinal axis, 2D trajectory, 3D trajectory; and thereby to propel the device through the medium.

According to some embodiments, one of the following holds true:
the medium comprises at least one material selected from: viscoelastic medium, extracellular matrix, interstitial space, biological compartment, biological duct, biological vessel, biological node, biological tissue, biological organ;
the screw-like element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
   at least one nickel-plated neodymium optionally selected from: N35, N38, N40, N42, N45, N48, N50, N52, and N55; or
   at least one alternative permanent nano/micro magnet material selected from: samarium cobalt (SmCo), alnico, ceramic, ferrite.

According to some embodiments,
the screw-like element comprises:
   length of ranging between 1.1-1.7 mm;
   outer diameter ranging between 0.57-0.65 mm;
   inner diameter ranging between 0.38-0.5 mm;
   pitch ranging between 0.34-0.60 mm;
   the hole diameter ranging between 0.2-0.4 mm;
the magnet comprises:
   diameter ranging between 0.2-0.5 mm;
   length ranging between 0.5-1.5 mm.

According to some embodiments of the invention, a propelling device is provided, configured to propel through a medium, using external magnetic stimuli applied thereon, the device comprising:
a propelling element comprising:
   a drill-bit-like element or a chisel-like, configured to vacate the surrounding medium as it rotates through; or
   a screw-like element, characterized by a cylindrical core and a helical ridge; or
   a twisted-ribbon-like element;
a cylindrical magnet, attached to the back end of the propelling element, wherein their longitudinal axes are aligned.

According to some embodiments, the diameter of the cylindrical magnet equals to—or smaller then—the outer diameter of the propelling element.

According to some embodiments, the magnet is attached to the back end of the propelling element via an adhesive material, optimally comprising at least one of: epoxy, acrylics, polyurethane, UV curable, and cyanoacrylate based materials.

According to some embodiments, the magnet is configured to respond to the applied magnetic stimuli and to rotate the propelling element; and wherein the propelling element is configured to convert rotary motion thereof into translation motion along at least one of: the longitudinal axis, 2D trajectory, 3D trajectory; and thereby to propel the device through the medium.

According to some embodiments, one of the following holds true:
the medium comprises at least one material selected from: viscoelastic medium, extracellular matrix, interstitial space, biological compartment, biological duct, biological vessel, biological node, biological tissue, biological organ;
the propelling element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
   at least one nickel-plated neodymium optionally selected from: N35, N38, N40, N42, N45, N48, N50, N52, and N55; or
   at least one alternative permanent nano/micro magnet material selected from: samarium cobalt (SmCo), alnico, ceramic, ferrite.

According to some embodiments,
the device comprises:
   length of ranging between 1.0-3.3 mm;
   propelling element's outer diameter ranging between 0.5-1.5 mm;
   propelling element's inner diameter ranging between 0.2-0.85 mm;
   propelling element's pitch ranging between 0.44-0.81 mm;
the magnet comprises:
   diameter ranging between 0.2-0.6 mm;
   length ranging between 0.5-1.5 mm.

According to some embodiments of the invention, a propelling device is provided, configured to propel through a medium, using external magnetic stimuli applied thereon, the device comprising:
a tube, characterized by a carved helical-like front section;
a cylindrical magnet, accommodated within the bore of the tube, at it's back section, wherein their longitudinal axes are aligned.

According to some embodiments, the magnet is configured to respond to the applied magnetic stimuli and to rotate the tube; and wherein the tube's carved helical-like front section is configured to convert rotary motion thereof into translation motion along at least one of: the longitudinal axis, 2D trajectory, 3D trajectory; and thereby to propel the device through the medium.

According to some embodiments, one of the following holds true:
the medium comprises at least one material selected from: viscoelastic medium, extracellular matrix, interstitial space, biological compartment, biological duct, biological vessel, biological node, biological tissue, biological organ;
the tube comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
   at least one nickel-plated neodymium optionally selected from: N35, N38, N40, N42, N45, N48, N50, N52, and N55; or
   at least one alternative permanent nano/micro magnet material selected from: samarium cobalt (SmCo), alnico, ceramic, ferrite.

According to some embodiments:
the tube comprises:
   length of ranging between 1.7-3.5 mm;
   outer diameter ranging between 0.76-0.83 mm;
   inner diameter ranging between 0.3-0.6 mm;
   pitch of the helical section ranging between 0.51-1.50 mm;
the magnet comprises:
   diameter ranging between 0.3-0.6 mm;
   length ranging between 0.5-3.0 mm.

According to some embodiments of the invention, a propelling device is provided, configured to propel through a medium, using external magnetic stimuli applied thereon, the device comprising:
a wedge-like element, configured to pierce through the medium as it translates through; and
a magnet, attached to the back end of the wedge-like element, wherein the magnet's longitudinal axis is parallel to the wedge-like element's back end wall.

According to some embodiments, the magnet is attached to the back end of the wedge-like-element via an adhesive material, optimally comprising at least one of: epoxy, acrylics, polyurethane, UV curable, and cyanoacrylate based materials.

According to some embodiments, the magnet is configured to respond to the applied magnetic stimuli and to translate the wedge-like-element, and thereby to propel the device through the medium.

According to some embodiments, one of the following holds true:
the medium comprises at least one material selected from: viscoelastic medium, extracellular matrix, interstitial space, biological compartment, biological duct, biological vessel, biological node, biological tissue, biological organ;
the wedge-like element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
   at least one nickel-plated neodymium optionally selected from: N35, N38, N40, N42, N45, N48, N50, N52, and N55; or
   at least one alternative permanent nano/micro magnet material selected from: samarium cobalt (SmCo), alnico, ceramic, ferrite.

According to some embodiments:
the wide-like-element comprises:
   side length ranging between 0.2-2.5 mm;
   height ranging between 0.2-5.0 mm;
   head angle ranging between 25-75 deg;
the magnet comprises:
   diameter ranging between 0.2-0.6 mm;
   length ranging between 0.2-3.0 mm.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 demonstrates an example for a propelling device having a helical spring-like element, according to various embodiments of the invention;

FIGS. 2A, 2B, 2C and 2D demonstrate four more examples for a propelling device having a helical spring-like element, according to various embodiments of the invention;

FIG. 3 demonstrates an example for a propelling device comprising a screw-like element, according to various embodiments of the invention;

FIGS. 4A, 4B, 4C and 4D demonstrate four examples for a propelling device having a magnet attached to a propelling element, according to various embodiments of the invention;

FIG. 5 demonstrates another example for a propelling device having a magnet attached to a propelling element, according to various embodiments of the invention;

FIG. 6 demonstrates an example for a propelling device having a carved helical section, according to various embodiments of the invention;

FIG. 7 demonstrates an example for a propelling device having a wedge-like element, according to various embodiments of the invention;

FIG. 8 demonstrates an example for a method of inserting a propelling device into an anesthetized rat's liver, according to various embodiments of the invention;

FIG. 9 demonstrates an example for a method and an apparatus configured for external stimuli and control of a propelling device, according to various embodiments of the invention;

FIG. 10 demonstrates an example for a use of the apparatus for external stimuli and control of a propelling device, according to various embodiments of the invention;

FIGS. 11A, 11B and 11C demonstrate test results for levels of representative liver enzymes (ALT, AST) at days 0, 1 and 14 post-treatment with SKC8 particle, according to various embodiments of the invention;

FIGS. 12A, 12B and 12C demonstrate test results for levels of representative liver enzymes (ALT, AST) at days 0, 1 and 14 post-treatment with Hovo2 particle, according to various embodiments of the invention;

FIGS. 13A, 13B, 13C, 13D and 13E demonstrate levels of representative liver enzymes (ALT, AST) at days 0, 1 and 14 post-treatment with Hovo2 particle and 20 G needle, according to various embodiments of the invention;

FIGS. 14A, 14B, 14C and 14D demonstrate images of liver damage of rat treated with Hovo2 microbot taken at 1 hr, 3 hr, 24 hr, and 14 days, respectively;

FIGS. 15A and 15B demonstrate liver injury score, observed in all sample's vs. time after treatment;

FIG. 16 demonstrates ultrasound image of spring basil microbot, processed using image tracking software; and

FIG. 17 demonstrates a retraction device, which uses an Eppendorf tube with an ND52 0.8 mm magnet located on the tip.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Reproducible and accurate propulsion of nano-/micro-particles in different biological matrices poses a formidable challenge. Controlled motion of micro robot (also noted as “microbot”) in a biologically or medically relevant environment depends on reliable external force, as well as on the properties of respective nano-/micro-particles of the microbot.

Both normal and pathological tissues exhibit distinct biophysical microscale features, posing specific requirements on the particle's Shape-, Size-, Surface- and material-properties (e.g. Stiffness)—(also noted as “4S properties”). The various embodiments of the currently provided invention are configured to answer these challenges, exhibiting varying 4S properties, as described in the following.

According to various embodiments of the present invention, a platform for active and accurate delivery of microparticles, is provided, endowed with diverse therapeutic load(s) and/or diagnostics to a specific location using external stimuli.

It is contemplated that propelling devices and microbots according to embodiments of the present invention will include particles described in International Patent Application PCT/US2018/030960 filed on May 3, 2018 and titled “METHODS AND SYSTEMS TO CONTROL PARTICLES AND IMPLANTABLE DEVICES,” which is hereby incorporated by reference in its entirety. Briefly, such particles are microelectromechanical (MEM) propelling devices, which comprise: (i) an actuator; (ii) a responsive element; (iii) a sensor; and (iv) an electronic circuit; wherein: said actuator controls and operates said responsive element; said electronic circuit controls said actuator; and said sensor receives signals transmitted by a remote unit. It is also contemplated that propelling devices and microbots according to embodiments of the present invention will be included in the platforms described in International Patent Application PCT/US2018/030960. Briefly, such platforms comprise the following modules: (a) one or more propelling devices or microbots described herein and comprising embedded logic and various MEM components; (b) a delivery and/or retraction module, configured to deliver and/or retract the devices; (c) an external signal generator; (d) an imaging module, configured to monitor said particles; and (e) an integration module configured to receive inputs from and to provide output control commands to other modules; wherein: said modules are configured to interact/communicate with each other; and said modules are internally controlled, externally controlled or both; and wherein said platform provides active, pre-determined, fully controlled, precise delivery of said devices in vitro, in vivo, and/or in a patient.

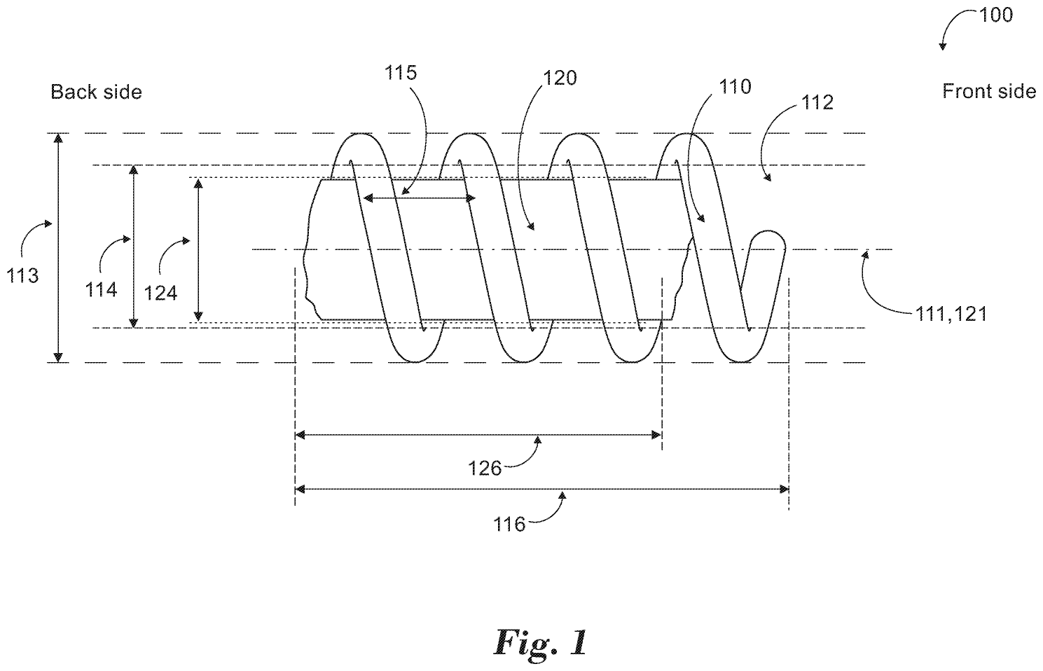

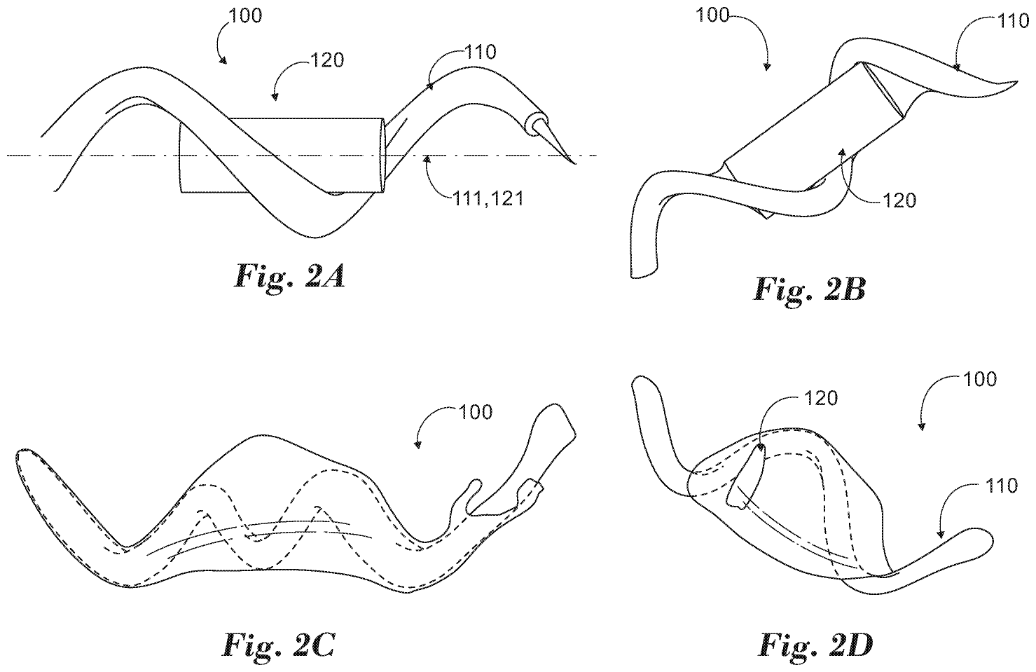

Reference is now made to FIGS. 1, 2A, 2B, 2C and 2D, which demonstrate (helical) “spring based” propelling microbots, as provided according to some embodiments of the invention, which are configured to provide a corkscrew-like motion, thereby an effective propulsion motion through varying viscoelastic media.

According to some embodiments, a propelling device (100) is provided, configured to propel through a medium, using external magnetic stimuli applied thereon, the device comprising:
a helical spring-like element (110); and
a cube, cuboid, prism, ellipsoid, disc-like, cylindrical magnet (120), accommodated within the helical element, wherein their longitudinal axes (111,121) are aligned.

According to some embodiments, the magnet is configured to respond to the applied magnetic stimuli and to rotate the helical element; and the helical element is configured to convert rotary motion thereof into a translation motion along at least one of: the longitudinal axis, 2D trajectory, 3D trajectory, and thereby to propel the device through the medium.

According to some embodiments, the medium (not shown), mentioned above and/or in the following, comprises at least one of: viscoelastic medium, extracellular matrix, interstitial space, biological compartment, biological duct, biological vessel, biological node, biological tissue, biological organ.

According to some embodiments, the front end of the helical element comprises a sharp and/or chiseled tip (112).

According to some embodiments, the magnet is accommodated at a center section of the helical element (as demonstrated in FIGS. 2A-2D), at a front section of the helical element (not shown), or at a back section of the helical element (as demonstrated in FIG. 1). It is noted that the terms “front” and “back” are relative to the designed motion direction of the microbot particle. According to some embodiments, the location and the length of the magnet is determined based on the medium to propelled in.

According to some embodiments, the magnet is fixed to the helical element, optionally via an adhesive material. According to some embodiments, the adhesive material comprises at least one of: epoxy, acrylics, polyurethane, UV curable, and cyanoacrylate based materials.

According to some embodiments, while the spring-based microbots provide efficient motion under ultrasonic images, due to the lack of its metallic components, their signal-to-noise ratio of ultrasonic responses may be improved. This may become problematic in vivo due to copious cavities present in organs of interest. According to some embodiments, a solution is provided by incorporating various diameters of mesoporous silica particles into the spring-based microbots.

According to such embodiments, the adhesive material is incorporated with mesoporous nano- or micro-silica particles, configured to enhance contrast under ultrasound radiation. Therefore, a “sonic spring based” microbot is provided.

An example for fabricating such a sonic spring based microbot includes a fabrication process which is nearly identical to that of spring-based microbots described above. Stainless steel micro-springs of inner diameters ranging from 0.4 mm to 1.1 mm with wire diameters ranging from 0.150 mm to 0.255 mm were extended with equal force on each end until the pitch of the spring was between 0.7 mm and 1.5 mm. Then, an end of this extended spring was clipped off with a nipper plier. Afterwards, an N52 magnet was inserted within the extended spring and axially aligned with the spring. A few milligrams (mg) of mesoporous silica particles were mixed in with epoxy. This mesoporous silica-incorporated epoxy was then applied uniformly around the magnet to affix it to the exterior spring. FIG. 2B demonstrates a typical sonic spring-based microbot. According to some further experiments, when the spring-based microbots were embedded with 1 μm mesoporous silica particles, there was a significant increase in brightness. This is due to the air-bubbles present in silica pore responding to the incident ultrasound.

According to some embodiments, in order to reduce variability in preparing the spring-based magnetic particles, microbots that do not require any adhesives, but fit snugly within the spring are provided herein. According to such embodiments, the magnet is encased with a layer of titanium vessel, before it is inserted into the helical element.

A non-limiting example is demonstrated in FIG. 1, showing an N52 magnet with an outer diameter of 0.5 millimeter (mm) and a length of 1 mm, which was encased in a thin layer of titanium vessel. Then, the titanium vessel containing the magnet was physically inserted inside the spring (110) with inner diameter of 0.61 mm and wire thickness (diameter) of 0.152 mm. The no-adhesive spring-based microbots were examined in freshly euthanized rat liver in vivo to exhibit good mobility under rotating magnetic field gradient. Subsequently, single microbot, presented in FIG. 1, traversed through various liver sub-compartments of eight rats at magnetic field strength of ˜250 mT and a gradient of 10 T/m without any damage or degradation.

According to some embodiments, at least part of the device is covered with—or embedded into a matrix that contains—an imaging agent, configured to facilitate its visualization ex vivo or in-vivo. The imaging agent optionally comprises at least one of: Rhodamine B, Fluorescein, microdefects, microbubbles, microdefects, mesoporous silica nano- and micro-particles, and Upconversion Phosphors.

According to some embodiments, the helical element comprises a material having Young's modulus stiffness above 1 Giga Pascal (GPa), optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals, titanium, titanium related alloys, stainless steel, gold.

According to some embodiments, the magnet (mentioned above and/or in the following) comprises:
at least one nickel-plated neodymium optionally selected from: N35, N38, N40, N42, N45, N48, N50, N52, and N55; or
at least one alternative permanent nano/micro magnet material selected from: samarium cobalt (SmCo), alnico, ceramic, ferrite.

According to some embodiments, the helical element comprises:
outer diameter (113) ranging between 0.66-1.2 mm;
inner diameter (114) ranging between 0.3-1.1 mm;
pitch (115) length ranging between 0.5-2.2 mm;
length (116) ranging between 1-5.6 mm.

According to some embodiments, the magnet comprises:
outer diameter (124) ranging between 0.3-0.8 mm;
length (126) ranging between 0.5-1.5 mm.

Examples for “Spring-Based” Microbots:

Stainless steel micro-springs of inner diameters, ranging from 0.4 mm to 1.1 mm with wire diameters ranging from 0.150 mm to 0.255 mm, were extended with equal force on each end until the pitch of the spring was between 0.7 mm and 1.5 mm. Then, an end of this extended spring was clipped off with a nipper plier. This created a sharp and chiseled tip for the microbot. Afterwards, a nickel-plated neodymium 52 (N52) magnet of varying diameters and lengths (diameters ranging from 0.3 mm to 0.8 mm and lengths ranging from 0.5 mm to 1.5 mm) were inserted within the extended spring and were axially aligned. The distance from the edge of the magnet to the tip of the spring was measured to be between 0.3 mm and 1.22 mm. Once the magnet was aligned with the spring's axis, it was fixed to the spring with a small amount of epoxy or cyanoacrylate and allowed it to cure for 8 hrs.

FIGS. 2A and 2B are representative images of spring-based particles, where the magnet was affixed to the spring with cyanoacrylate and epoxy, respectively. It was demonstrated that microbots fixed with epoxy tend to have a more rounded body than those glued with cyanoacrylate.

To aid the process of ex vivo and in vivo injection of the microbots into tissue, where there is limited visibility, various imaging agents were incorporated onto the microbots. When the magnets were glued to the springs using cyanoacrylate, the imaging agent (e.g. Rhodamine B, Fluorescein, Upconversion Phosphors) was first dusted on top of the magnet and afterwards cyanoacrylate was deposited on top to seal the imaging agents to the magnet. This process was repeated three times and after the deposition of third layer, final layer of cyanoacrylate was deposited. For the microbots glued with epoxy, the imaging agents were added to the epoxy mixture and mixed in prior to applying it on the microbot.

FIG. 2C is a representative image of spring-based microbots that has been dusted with Rhodamine B prior to application of cyanoacrylate. Likewise, FIG. 2D shows a spring-based microbot that has been affixed with Rhodamine B-suspended epoxy.

Particle propulsion was tested using both uniform (0.1 T) and gradient-based magnetic devices in order to select best performing system and particles.
(i) In Agar Under Gradient, Rotating Magnetic Field. Spring-based microbots were inserted into a piece of agar and subsequently placed at the predetermined distance away from the magnetic surface of the propulsion device (ca. 17-20 mm away from the center of the magnet in the gradient magnetic system), where the field strength is measured to be around 340 mT and its gradient 12 T/m. Upon application of magnetic field rotation of ˜1 Hz, rotation of movement of spring-based microbots were observed and their travel time recorded to calculate the average speed. 97% of spring-based microbots successfully traversed through agar and the average speed was 0.7 mm/sec.
(ii) In Agar Under Uniform, Rotating Magnetic Field. The mobility test results of the spring-based microbots showed varying results. The microbots that moved most efficiently in rotating uniform field tend to move faster than average spring-based microbots in rotating gradient field as it is demonstrated in Table 1.
(iii) In Vivo Rat Liver, Rotating Gradient Magnetic Field. One spring-based microbot (“SKC8”) traversed through four different rats' livers in vivo for the purpose of preliminary safety testing of the spring-based microbots. Based on injection position and retraction point, positive movement was confirmed under magnetic field strength of 280 mT and a gradient of 8 T/m.

TABLE 1
Summary of mobility tests of spring-based microbots

| | | Success | Average | Field | |
|---|---|---|---|---|---|
| | | Rate | Speed | Strength | Gradient |
| Field type | Medium | (%) | (mm/s) | (T) | (T/m) |
| Uniform | Agar | 33.33333333 | 1.559 | 0.1 | 0 |
| Rotating | | | | | |
| Rotating | Agar | 96.66666667 | 0.724 | 0.338 | 12 |
| Gradient | | | | | |
| Uniform | Rat liver | 0/0 | N/A | N/A | N/A |
| Rotating | in vivo | | | | |
| Rotating | Rat liver | 4/4 | No info | 0.28 | 8 |
| Gradient | in vivo | | | | |

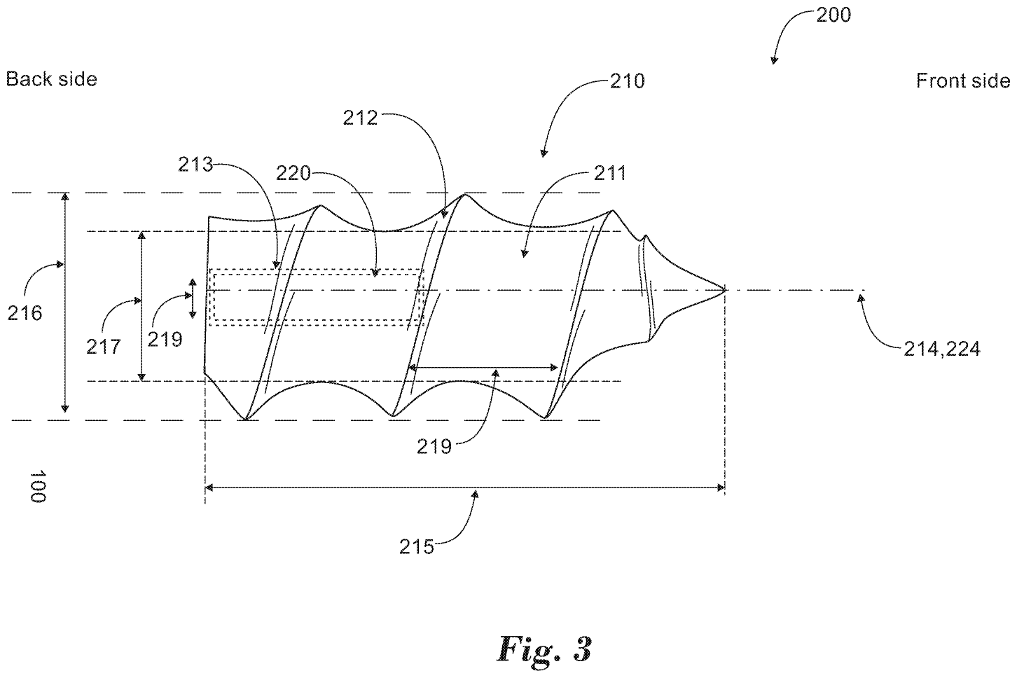

Reference is now made to FIG. 3, which demonstrates a “screw-shaped” propelling microbot, provided according to some embodiments of the invention. According to some embodiments, this configuration closely mimics the shape of a screw, with a sharp tip and a base with constant pitch. The screw-shape microbot configured to:
reduce the total length of the microbot, thereby minimize damage that may be caused to the organ of interest; and
produce a screw-like motion that aids with propulsion in vivo.

According to some embodiments, a propelling device (200) is provided, configured to propel through a medium, using external magnetic stimuli applied thereon, the device comprising:
a screw-like element (210), characterized by a conical-core (not shown) or a cylindrical-core (211) and a helical ridge (212);
a cylindrical magnet (220), accommodated within a hole (213) drilled in the cylindrical core, wherein their longitudinal axes (214,224) are aligned.

According to some embodiments, the magnet is accommodated at a back section of the cylindrical core. According to some embodiments, the magnet is provided at a front section of the cylindrical core (not shown), in such embodiments, the length of the provided magnet is smaller than the length of the drilled hole.

According to some embodiments, the magnet is configured to respond to the applied magnetic stimuli and to rotate the helical element; and the screw-like element is configured to convert rotary motion thereof into translation motion along at least one of: the longitudinal axis, 2D trajectory, 3D trajectory; and thereby to propel the device through the medium.

According to some embodiments, the screw-like element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold.

According to some embodiments, the screw-like element comprises:
length (215) of ranging between 1.1-1.7 mm;
outer diameter (216) ranging between 0.57-0.65 mm;
inner diameter (217) ranging between 0.38-0.5 mm;
pitch (218) ranging between 0.34-0.60 mm;
the hole diameter (219) ranging between 0.2-0.4 mm.

According to some embodiments, the magnet (220) comprises:
outer diameter ranging between 0.2-0.5 mm:
length ranging between 0.5-1.5 mm.

Example for “Screw-Based” Microbot:

A screw-like gold casing was fabricated with a length of 1.5 mm and total width (diameter) of 0.54 mm. The pitch of the screw was measured to be 0.39 mm. Afterwards, a small hole with a diameter of 0.3 mm was drilled into the screw end. An N52 magnet with a diameter of 0.3 mm and length of 1 mm was inserted into the hole. A representative image of such a screw-shaped microbot is provided in FIG. 3.

Under rotating a magnetic field gradient, when the screw-shaped microbots were embedded in agar, the microbots traveled at a comparable speed as those of spring microbots in agar under similar conditions. When they were inserted in freshly euthanized rat liver, however, the propulsion was slower as compared to the spring-based particles.

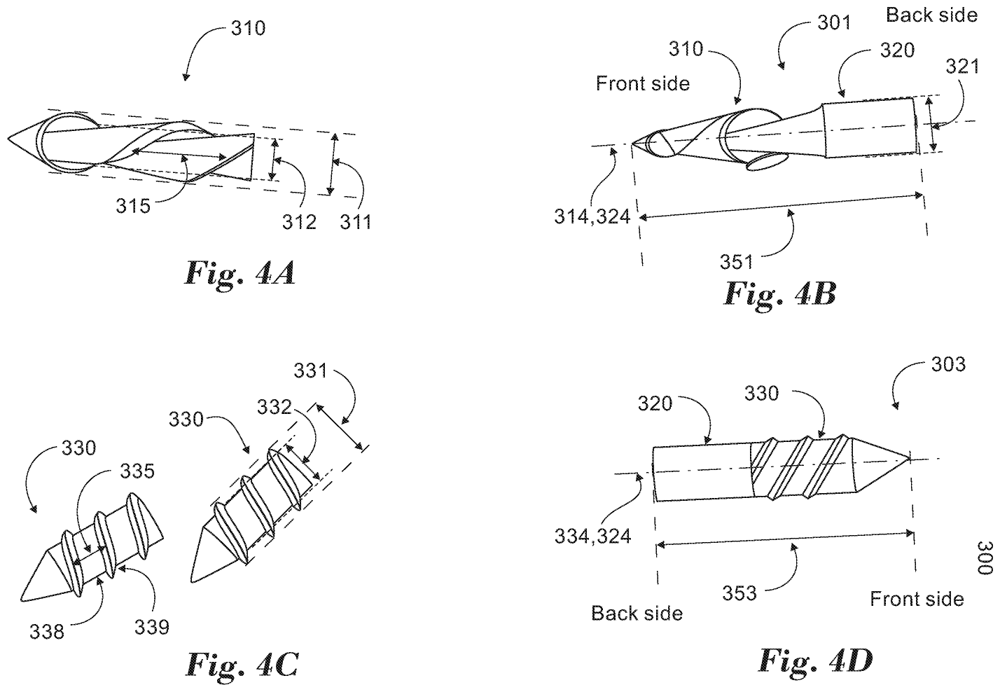

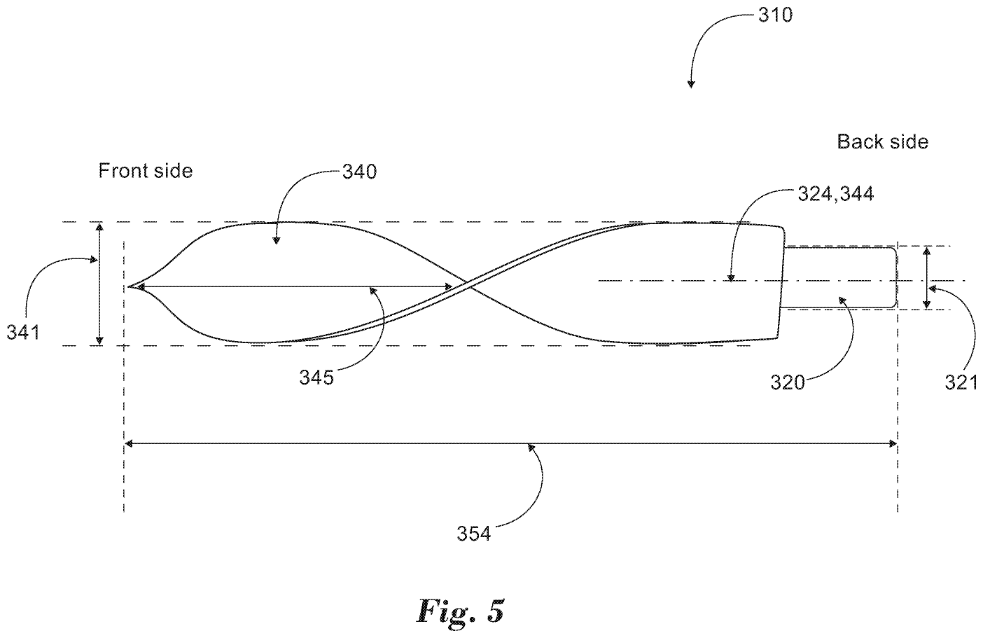

Reference is now made to FIGS. 4A-4D and FIG. 5, which demonstrate propelling microbots comprising a magnet attached to a propelling element, provided according to some embodiments of the invention.

Microdrill bits are configured to provide a unique topology optimized to vacate the surrounding medium as they rotate through. Due to the limited inner diameter of the drill bit core, largest hole that can be drilled without compromising the integrity is about 0.1 mm, which may be too small to insert any magnets. Therefore, a provided solution is to attach a magnet at the base of the microdrill bit.

According to some embodiments, a propelling device (301,304,305), is provided configured to propel through a medium, using external magnetic stimuli applied thereon, the device comprising:
a propelling element comprising:
   a drill-bit-like element (FIGS. 4A-4B, 310) or a chisel-like (not shown), configured to vacate the surrounding medium as it rotates through; or
   a screw-like element (FIGS. 4C-4D, 330), characterized by a cylindrical core (338) and a helical ridge (339); or
   a twisted-ribbon-like element (FIG. 5, 340);
a cube, cuboid, prism, ellipsoid, disc-like, cylindrical magnet (320), attached to the back end of the propelling element, wherein their longitudinal axes (314/334/344,324) are aligned.

According to some embodiments, the diameter (321) of the cylindrical magnet equals to—or smaller then—the outer diameter (311/331/341) of the propelling element.

According to some embodiments, the magnet is attached to the back end of the propelling element via an adhesive material, optimally comprising at least one of: epoxy, acrylics, polyurethane, UV curable, and cyanoacrylate based materials.

According to some embodiments, the magnet is configured to respond to the applied magnetic stimuli and to rotate the propelling element; and wherein the propelling element is configured to convert rotary motion thereof into translation motion along at least one of: the longitudinal axis, 2D trajectory, 3D trajectory; and thereby to propel the device through the medium.

According to some embodiments, the propelling element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold.

According to some embodiments, the device (301,304,305) comprises:
length (351,353,354) of ranging between 1.0-3.3 mm;
propelling element's outer diameter (311,331,341) ranging between 0.5-1.5 mm;
if relevant, propelling element's inner diameter (312,332) ranging between 0.20-0.85 mm;
propelling element's pitch ranging (315,335,345) between 0.44-0.81 mm.

According to some embodiments, the magnet (320) comprises:
outer diameter ranging between 0.2-0.6 mm;
length ranging between 0.5-1.5 mm.

Examples for “Drill-Bit/Screw-Like” Microbots (with Back Attached Magnets):

N52 magnets with a diameter of 0.6 mm and length of 1 mm were attached to two different types of tips (propelling elements). First, various configurations of microdrill bits were purchased and sent for post processing to laser-cut the drill bit tips to lengths of 2 mm. A representative image of these microdrill tip is provided in FIG. 4A. A second set of tips were fabricated to 1.5 mm long micro-screws with outer diameter of 0.75 mm and pitches of either 2 turns/mm or 3 turns/mm (FIG. 4C).

A piece of N52 magnet with an outer diameter of 0.6 mm and length of 1 mm was dipped into epoxy and was fixed to the base of either the microdrill bit tips and the micro-screw tips and held together by hand for a couple of minutes until they remained stationary. Afterwards, the microbots were left in air overnight for curing. Representative images of microbots fabricated with micro-drill bit tips and micro-screw tips are presented in FIG. 4B and FIG. 4D, respectively.

Two drill-bit-based microbots that had been assembled thus far, exhibited slower movement in agar under rotating gradient field (under 0.05 mm/s) as compared to the spring-based particles.

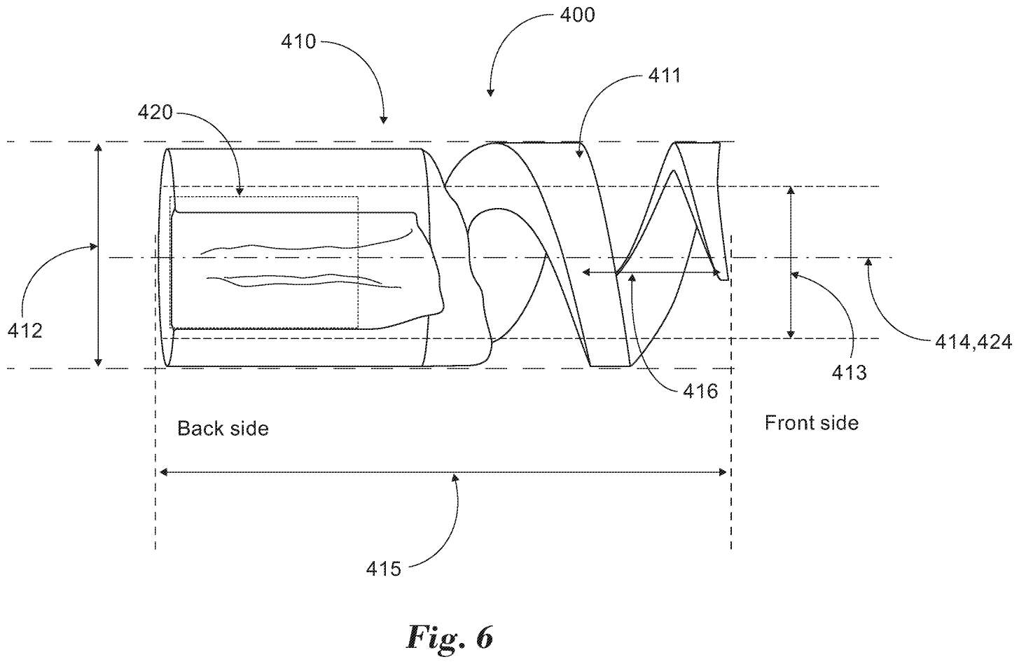

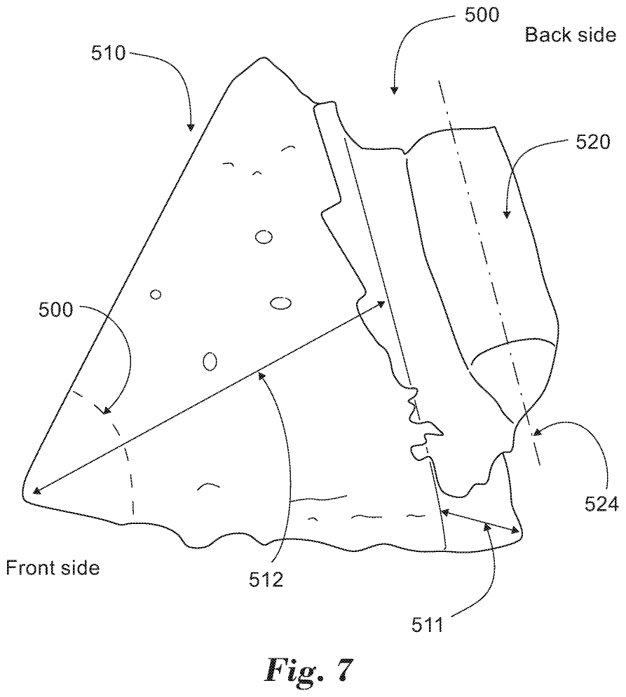

Reference is now made to FIG. 6, which demonstrates a “carved helix” propelling, provided according to some embodiments of the invention.

According to some embodiments, by carving out a hollow metal tube (including but not limited to titanium and stainless steel) into a helical shape, it allows one to control various physical parameters such as pitch and wire thickness. Furthermore, the use of thicker helices, provides more rigidity to the helices, thereby provides more support during propulsion.

According to some embodiments, a propelling device (400) is provided, configured to propel through a medium, using external magnetic stimuli applied thereon, the device comprising:
a tube (410), characterized by a carved helical-like front section (411);
a cube, cuboid, prism, ellipsoid, disc-like, cylindrical magnet (420), accommodated within the bore of the tube, at its back section, wherein their longitudinal axes (414,424) are aligned.

According to some embodiments, the magnet is configured to respond to the applied magnetic stimuli and to rotate the tube; and wherein the tube's carved helical-like front section is configured to convert rotary motion thereof into translation motion along at least one of: the longitudinal axis, 2D trajectory, 3D trajectory; and thereby to propel the device through the medium.

According to some embodiments, the tube comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold.

According to some embodiments, the tube comprises:
length (415) of ranging between 1.7-3.5 mm;
outer diameter (412) ranging between 0.76-0.83 mm;
inner diameter (413) ranging between 0.3-0.6 mm;
pitch (416) of the helical section ranging between 0.51-1.50 mm.

According to some embodiments, the magnet (420) comprises:
outer diameter ranging between 0.3-0.6 mm;
length ranging between 0.5-3.0 mm.

An example for a carved-helix microbot is provided in FIG. 6, where an N52 magnet (hidden, 420) was inserted into the base of a metallic tube (410) with an inner diameter that matches the outer diameter of the magnet. Subsequently, the tip of the metallic tube was carved out (411) with a metal cutting device such as diamond tip cutter, laser, CNC tool, and other micro-cutting techniques to create helices. A representative image of the carved-helix microbot (400) is provided in FIG. 6.

A representative carved-helix microbot was tested and traveled at a speed of ˜0.5 mm/s in agar; notes: (1) the speed was estimated and not rigorously measured, (2) the test was conducted with Macho 3.0 and therefore cannot be directly compared with speeds of other microbots. According to the test's results, the tube's length and the ratio between the helical section and smooth magnetic casing influences on microbot mobility.

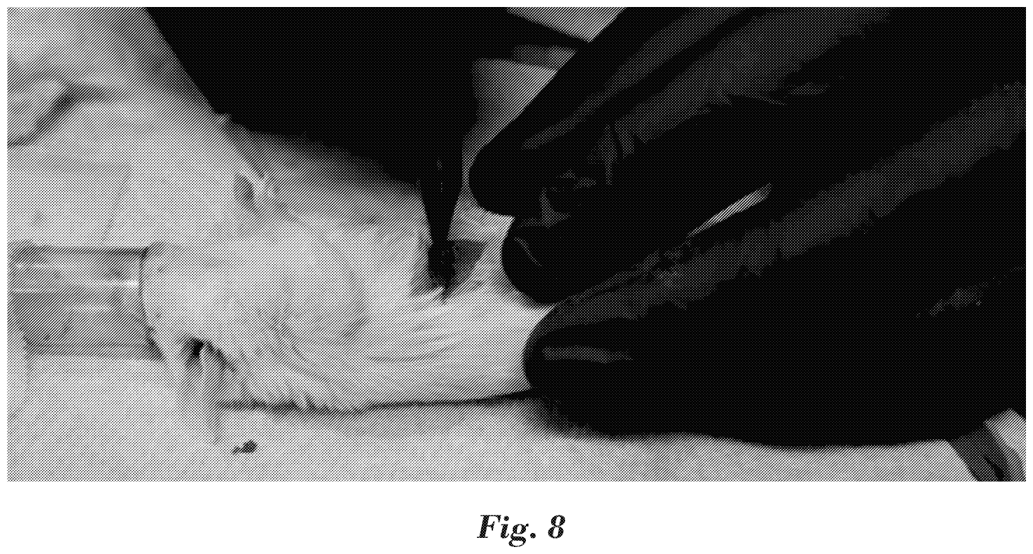

According to some embodiments, and as demonstrated in FIG. 7, a propelling device (500) is provided, configured to propel through a medium, using external magnetic stimuli applied thereon, the device comprising:
a wedge-like element (510), configured to pierce through the medium as it translates through; and
a magnet (520), attached to the back end of the wedge-like element, wherein the magnet's longitudinal axis (524) is parallel to the wedge-like element's back end wall.

According to some embodiments, the magnet is attached to the back end of the wedge-like-element via an adhesive material, optimally comprising at least one of: epoxy, acrylics, polyurethane, UV curable, and cyanoacrylate based materials.

According to some embodiments, the magnet is configured to respond to the applied magnetic stimuli and to translate the wedge-like-element, and thereby to propel the device through the medium.

According to some embodiments, the wedge-like element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold.

According to some embodiments, the wide-like-element (520) comprises:
side length (511) ranging between 0.2-2.5 mm;
height ranging (512) between 0.2-5.0 mm;
head angle (513) ranging between 25-75 deg;

According to some embodiments, the magnet (520) comprises:
outer diameter ranging between 0.2-0.6 mm;
length ranging between 0.2-3.0 mm.

According to some embodiments, the above-mentioned magnets can comprise a cylinder, a ring, or a tube configuration. It is noted herein that when the “outer” diameter of the magnet is referred to, it may also refer to a cylinder's diameter; or if the magnet's diameter is mentioned, if relevant it may refer to the outer diameter.

### Microbots Dimension Examples

The above-mentioned propelling devices were test with various shapes and surfaces. Further, within same shape and surface properties, various dimensions were tested to demonstrate the effects that physical dimensions have on microbot mobility. Table 2 provides the dimension ranges per class of microbot.

TABLE 2
range of dimensions

| | Length | Outer Diameter | Inner Diameter | Pitch |
|---|---|---|---|---|
| Bot Type | (mm) | (mm) | (mm) | (mm) |
| Spring-Based | 1.4-5.6 | 0.66-1.2 | 0.41-1 | 0.67-2.17 |
| Microbots | | | | |
| Screw-Shaped | 1.1-1.7 | 0.57-0.65 | 0.38-0.45 | 0.34-0.6 |
| Microbots | | | | |
| Prefabricated | 2.6-3.3 | 0.71-0.83 | 0.41-0.64 | 0.44-0.81 |
| Bits | | | | |
| No Adhesive | 1.7-4.2 | 0.94-1.3 | 0.67-0.83 | 0.43-0.82 |
| Spring-Based | | | | |
| Microhots | | | | |
| Carved Helix | 1.7-3.4 | 0.76-0.83 | 0.50-0.55 | 0.51-1.5 |
| Microbots | | | | |
| Sonic | 1.4-5.6 | 0.66-1.2 | 0.41-1 | 0.67-2.17 |
| Sprin-Based | | | | |
| Microbots | | | | |

| | Side Length | Height | Head Angle |
|---|---|---|---|
| | (mm) | (mm) | (deg) |
| Wedge-Shaped | 2.2-2.5 | 2.6 | 68 |
| Microbots | | | |

### Tests and Results

Highly localized and patient-specific treatment of multiple diseases, including cancer is an emerging strategy due to its enhanced clinical efficacy-safety outcome. A platform was provided for active and accurate delivery of microparticles endowed with diverse therapeutic load(s) and/or diagnostics to a specific location using external stimuli. In these tests, tissue damage was investigated, caused by representative devices or a needle (positive control) in vivo. The propulsion of the currently provided particles through the liver tissue was safe and well tolerated in both mice and rats (N=40 total). No significant differences were detected in the livers of animals treated with the needle or the current particles. Notably, all treated rodents (mice/rat) survived and behaved normally liver recovery was observed and confirmed complete, by day 14 post-treatment using both histology and representative biomarkers (ALT/AST).

The ability to deliver drugs through diverse heterogeneous tissue in a highly controlled and safe manner both spatially and longitudinally is anticipated to enhance safety-efficacy profile of multiple therapeutic agents and to address patient-specific conditions. The focus of the currently provided technology is active and precise delivery of diverse therapeutics and/or diagnostics agents to a tissue of interest including liver. The technology is likely to become a standalone approach or supplement the existing standards of care suitable for the treatment of localized conditions including but not limited to tumors, inflammation, chronic pain, eye and/or muscle degenerative disorders and bacterial infections. Towards this goal, a multimodal platform was developed that includes magnetic propulsion, versatile microparticles, imaging-/image-analysis and particle delivery and retraction modules. Notably, the provided particle is capable of delivering diverse payloads including drugs and diagnostics, both small molecules and biologics to remote, hard to reach locations in the human body in a minimally invasive manner. The currently provided microparticles (microbots) can move with high degree of accuracy in a variety of biological media including liver, gastric, vitreous tissues and deliver diverse targeted payloads to treat affected areas of up to 7 cm³ in volume using a single device.

In the initial proof-of-concept studies, hepatocellular carcinoma (HCC) were selected as the therapeutic focus. HCC is the most common type of primary liver cancer in adults and is the most common cause of death in people with liver cirrhosis. While there are a number of accepted cytotoxic drugs (e.g., doxorubicin), targeted drugs (e.g., Nexavar™), as well as novel immuno-oncology therapies (e.g., Ipilimumab™), these drugs rarely result in durable benefit and/or may result in serious systemic side effects. While several drug delivery systems have been proposed and developed, these agents are still administered systemically and have not met their potential due to multiple challenges including inadequate efficacy.

In the series of experiments described herein, preliminary safety studies were conducted in rodents (N=40, 36 rats and 4 mice) aimed at assessment of the general liver safety/toxicity associated with the particle motion through the organ. Specifically, two distinct representative microbots were used to propel through the designated liver compartments, analyzed time-dependent (1 hr, 3 hrs, 24 hrs and 14 days) liver damage and compared it to positive control animal group treated with injection needle.

The examined data suggests that the currently provided particles traversed reliably, reproducibly and safely through the liver without causing general tissue damage. Longitudinal studies of the liver toxicity further indicate that both needle and microparticle treatment caused rapid and transient changes to the liver histology 3 hrs post-treatment. The pathology was dramatically reduced by day 7 and the liver tissue was completely recovered at day 14 post-treatment. These acute changes and recovery of the liver tissue by day 14 were further corroborated via measurements of representative blood biomarkers ALT and AST.

Visualizing and tracking movement of the microbots, through the liver, to estimate the accuracy of propulsion, were enabled using ultrasound imaging. Moreover, a prototype device was also designed, which is suitable for safe and accurate delivery and retraction of the particles, and these experiments were validated in both ex vivo and in vivo in murine models.

The primary purpose of the study was to evaluate the ability for the provided particle to move through a heterogeneous tissue (the liver) without causing non-transient toxicity.

Primary test goals of the study:
a. to propel a particle through organ/tissue of therapeutic interest (e.g., liver) in vivo using external magnetic stimuli followed by histopathological assessment of tissue damage;
b. to examine the initial time course (14 days) of tissue regeneration post-traumatic treatment with the particle(s) vs. needle (same outer diameter) as a positive control;

Secondary test goals of the study:
c. to visualize and track movement of the devices through tissue such as liver.
d. to determine distance traveled by the particle.
e. to develop a protocol for preliminary particle insertion-retraction protocol suitable for further optimization.

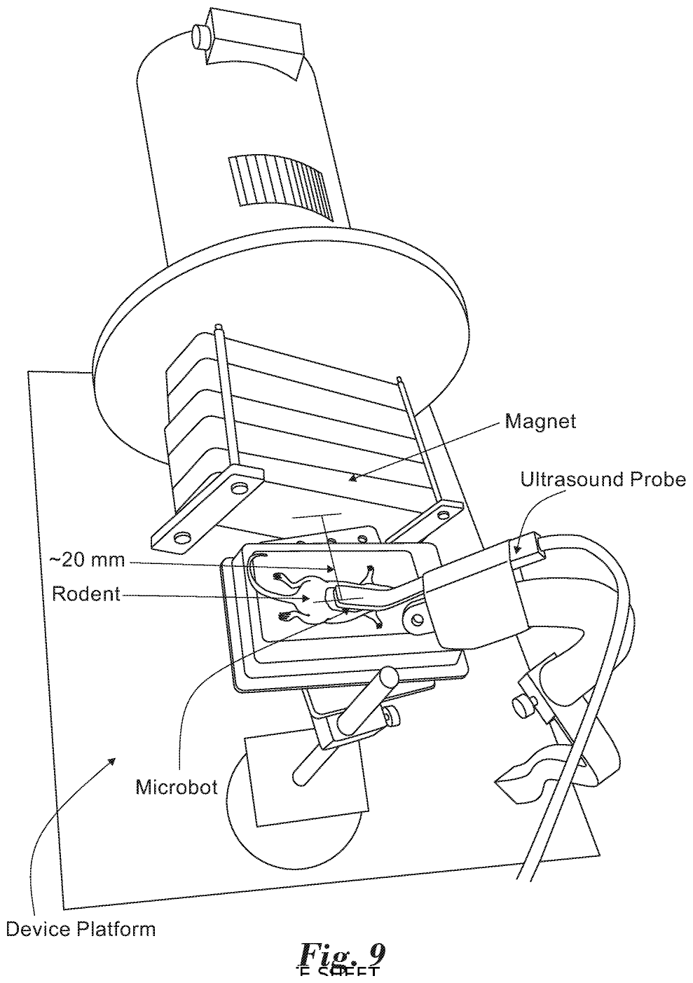

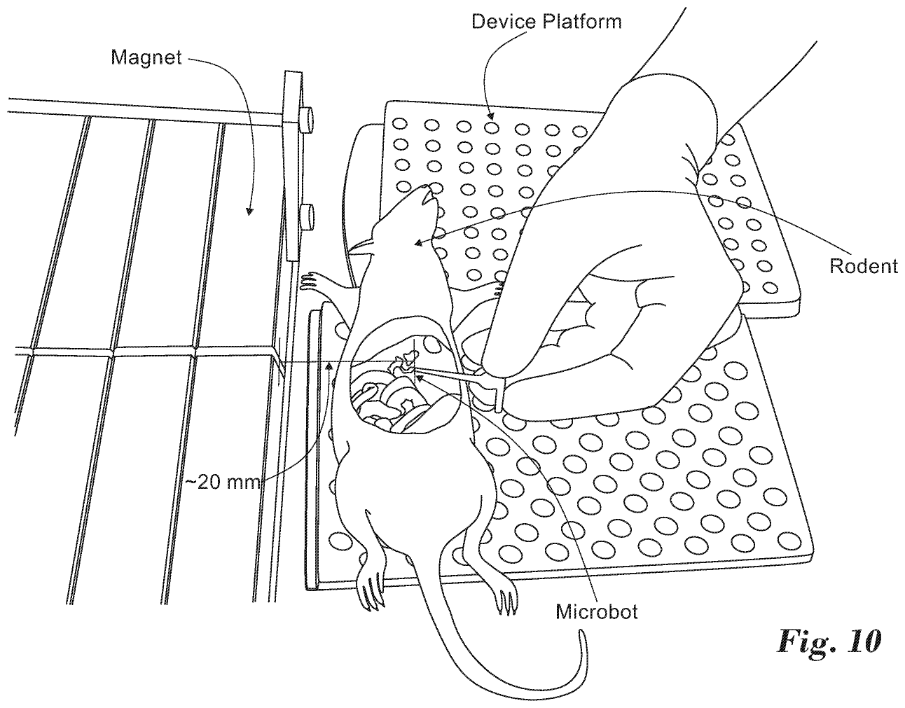

Procedure:
a) Animals: 6-8 weeks old female Sprague-Dawley (SD) rats (N=36) and 10 weeks old male BALBc mice (N=4).
b) Administration of Anesthesia (Isoflurane): The rat was anesthetized using 5% isoflurane in 100% O₂ with sedation to be confirmed with a toe pinch. The anesthesia was maintained at 1-2% isoflurane by inhalation and ventilation throughout the procedure. The surgical area was prepared by shaving and removing hair (if needed), cleansing the skin by wiping with 70% ethanol.
c) Intra-hepatic Implantation of the particle: Following anesthesia induction, a midline incision was made in the skin of the abdomen and a second incision was made into the peritoneal cavity using blunt scissors. Insertion and retraction of the particle was performed on the surgical table. A particle was inserted completely into either Right Medial Lobe or Left Lateral Lobe of the liver using plastic forceps. FIG. 8 demonstrates particle insertion in the liver (right medial lobe) of anesthetized rat using plastic forceps. Needle (20 G, ca. 0.91 mm outer diameter) puncture was used as a positive control to assess the liver damage. The puncture was performed via the open-wound procedure to emulate the particle insertion sequence or in situ through skin.
d) External Rotating Magnets: After inserting the particle, the anesthetized rat was moved to a device platform next to the external magnets. FIGS. 9 and 10 demonstrate an external propulsion platform, based on rotating permanent magnets; demonstrated are: the rotating magnets' set-up; the anesthetized animal; a platform for the animal; and an US probe. The position of the rat was adjusted so that the inserted particle is facing the center of the magnet at a predetermined distance (˜20 mm) using the proprietary fixed magnets platform (as in FIGS. 9 and 10). The particle was initiated and propelled using the external rotating magnets while being continuously observed as the device traversed the liver. Once the particle was ready to exit the liver as evidenced via visual observation, the rotating magnet was stopped. The animal was moved to the surgical table to retrieve the particle using Nd52 micromagnet (0.8×2 mm) attached to a plastic holder. FIG. 10 demonstrates relative position of the rat to the magnet; shown are: the surface of magnetic set-up, and approximate particle position. The distance traveled by the particle in the liver was measured using calipers (5-8 mm on average). The peritoneal cavity was closed post-procedure using nylon or polypropylene sutures. The animal was returned to the individual cage to recover with ad libitum access to food and water. Generally, the recovery from anesthesia took 25-30 min. All animals were monitored every 15 min post-surgery for ca. 3 hrs to ascertain overall well-being and normal physiological behavior. Notably, no animals were lost due to the procedure in either control (needle) or particle test groups. Moreover, all test animals seemed to have recovered completely within 1 hr post procedure.

### Study Design, Group 1:

TABLE 3

| | | | Bleeds on | Day 14 |
|---|---|---|---|---|
| No. | #Animals | Particle | Day 0, 1 and 14 | Liver Histology |
| 1 | 4 | SKC8 | Yes | Yes |
| | | (FIG. 2C) | | |
| 2 | 3 | Hovo2 | Yes | Yes |
| | | (FIG. 1) | | |

Blood and Liver Tissue Collection:

On Day 0, Day 1 and Day 14 post-procedure blood was collected by tail vein for measuring representative liver enzymes (ALT and AST, selected as dynamic markers based on the initial calibration studies). On Day 14, a specific liver tissue was collected (area traversed by the particle) for histology (H&E, hematoxylin and eosin staining)

### Study Design, Group 2:

TABLE 4

| | Needle/ | Time | | # | Bleeds on | Liver |
|---|---|---|---|---|---|---|
| # | Particle | Point | | Animals | Day 0, 1 and 14 | Histology |
| 1 | Hovo2 | 1 | hr | 3 | No | Yes |
| 2 | 20 G needle | 1 | hr | 3 | No | Yes |
| | open wound | | | | | |
| 3 | Hovo2 | 3 | hrs | 4 | No | Yes |
| 4 | 20 G needle | 3 | hrs | 3 | No | Yes |
| | open wound | | | | | |
| 5 | Hovo2 | 24 | hrs | 4 | No | Yes |
| 6 | 20 G needle | 24 | hrs | 4 | No | Yes |
| | open wound | | | | | |
| 7 | Hovo2 | 14 | days | 5 | Yes | Yes |
| 8 | 20 G needle | 14 | days | 3 | Yes | Yes |
| | open wound | | | | | |

Liver Tissue Collection:

1 hr, 3 hrs, 24 hrs and 14 days post-procedure, liver tissue (area traversed by the particle) was collected for histology (H&E, hematoxylin and eosin staining).

Results for Diverse Particles (SKC8 and Hovo2):

In the toxicity assessment tests, several representative particles were used that illustrate at least two diverse designs including ‘string based particle’ with a magnet accommodated in the center of the spring (SKC8), as demonstrated in FIG. 2C and ‘spring based particle’ with a magnet accommodated in the back of the spring (Hovo2), as shown in FIG. 1. Particles design and dimensions are summarized in Table 5.

TABLE 5

| | Length | Outer Diameter | Inner Diameter | Pitch |
|---|---|---|---|---|
| Particle | (mm) | (mm) | (mm) | (mm) |
| Hovo2 (FIG. 1) | 2.0 | 0.97 | 0.57 | 0.5 |
| SKC8 (FIG. 2C) | 2.8 | 0.66 | 0.38 | 1.6 |

SKC8 Blood ALT and AST Levels on Day 0, 1 and 14:

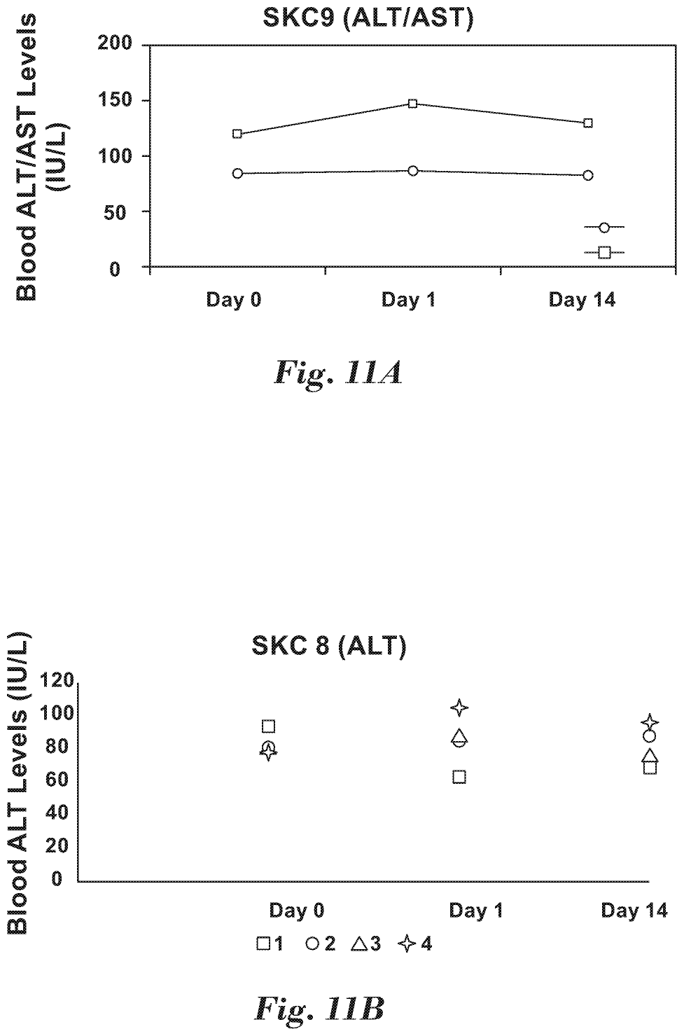

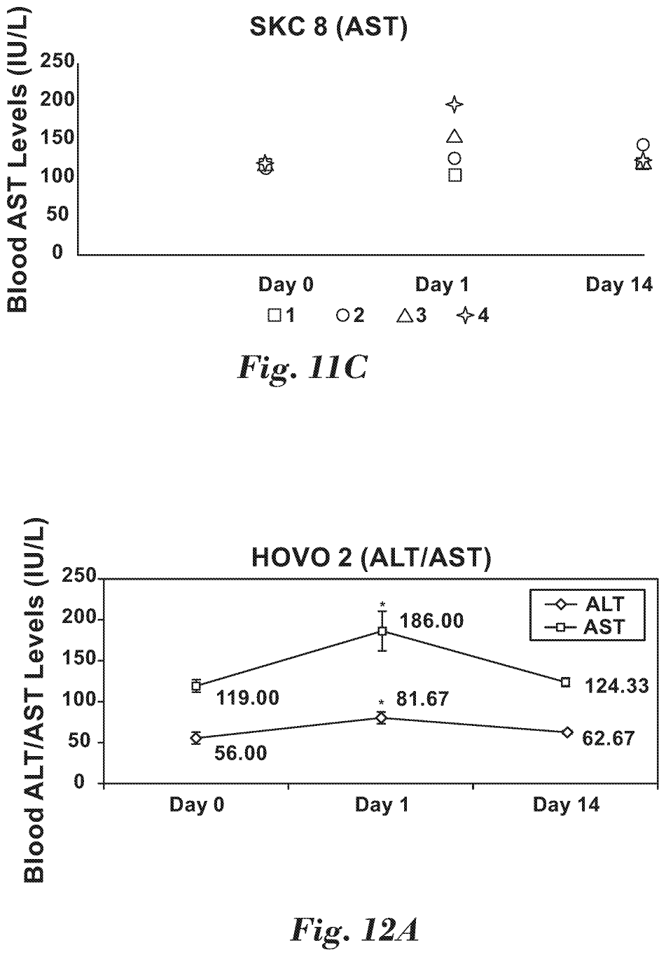

No significant changes were observed in blood ALT and AST levels over 14 days following the particle insertion in control vs particle treated animals AST levels were transiently elevated day 1 post-procedure but returned to normal at day 14. FIGS. 11A-11C demonstrate levels of representative liver enzymes (ALT, AST) at days 0, 1 and 14 post-treatment with SKC8 particle; FIG. 11A demonstrates overall profile of liver enzymes, FIGS. 11B and 11C demonstrate individual profile for the liver enzymes ALT and AST, respectively, where each differently colored circle represents an individual animal, N of 4 animals.

Hovo2 Blood ALT and AST Levels on Day 0, 1 and 14:

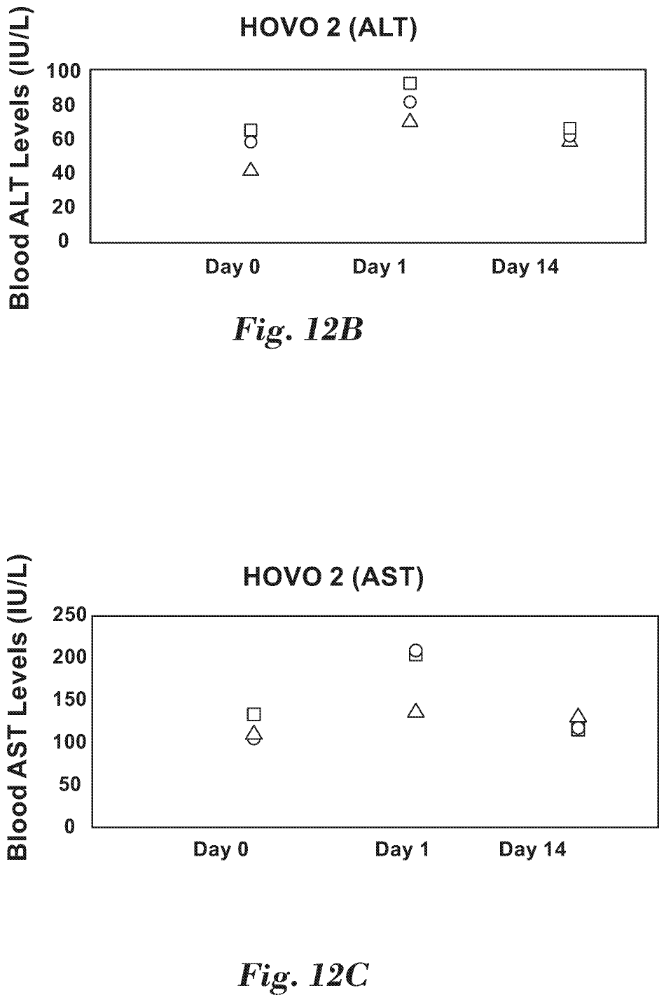

For the Hovo2 particle propulsion studies, significant changes in both ALT (56 vs. 82 IU/L) and AST (119 vs. 186 IU/L) were observed on Day 1 following particle insertion. On day 14, ALT and AST levels were similar to day 1 levels and were not significant. FIGS. 12A-12C demonstrate test results for levels of representative liver enzymes (ALT, AST) at days 0, 1 and 14 post-treatment with Hovo2 particle; FIG. 12A demonstrates overall profile of liver enzymes; FIGS. 12B and 12C demonstrate individual profile for the liver enzymes ALT and AST, respectively, where each differently colored circle represents an individual animal, N of 3 animals.

Time Course Study—Hovo2 vs. Needle Controls Blood ALT and AST Levels on Day 0, 1 and 14:

As anticipated, in the Hovo2 and the positive control experiments (using 20 G needle, internal diameter of 0.6 mm, outer diameter of 0.91 mm), blood values for ALT and AST were higher on Day 1 compared to Day 0, presumably due to acute local trauma (Ogawa et al, Healing of focal injury in rat liver. American Journal of Pathology (1985) 119: 158-167).

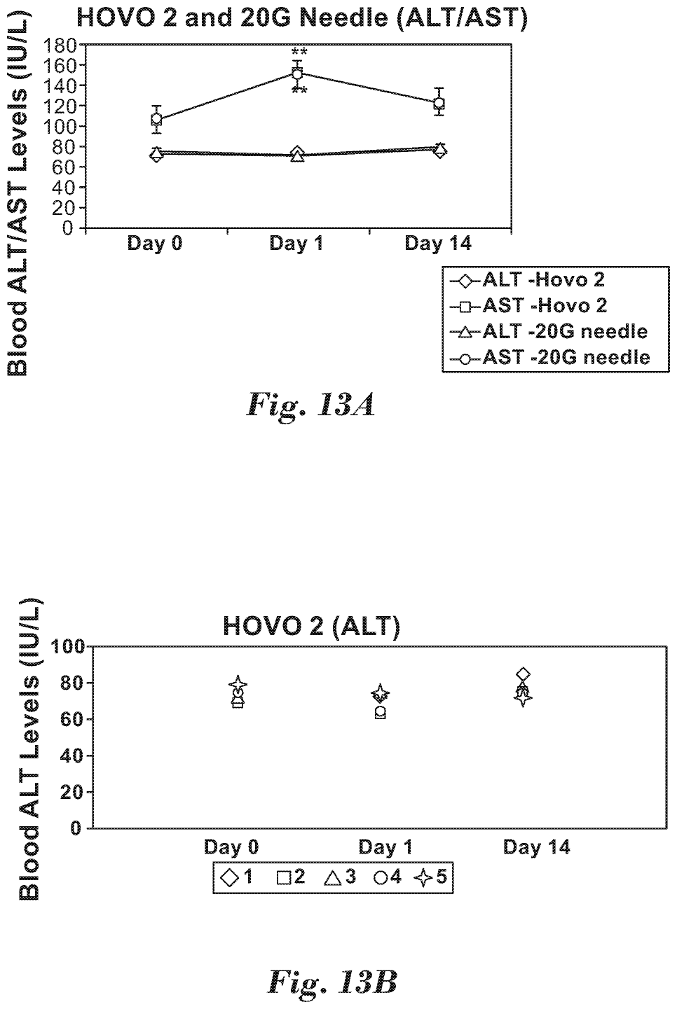

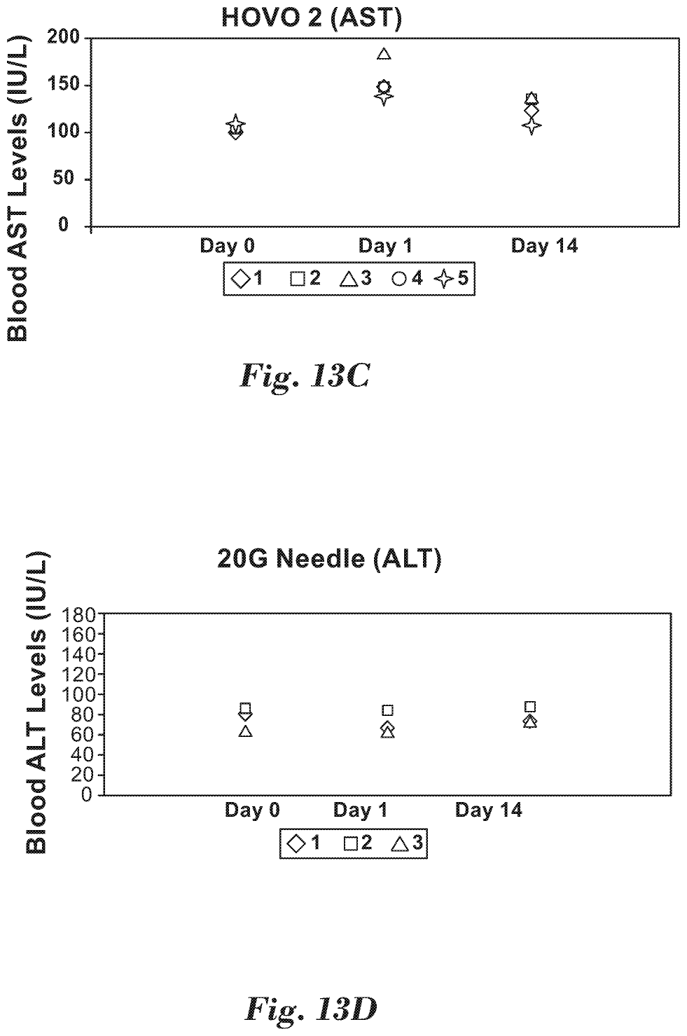

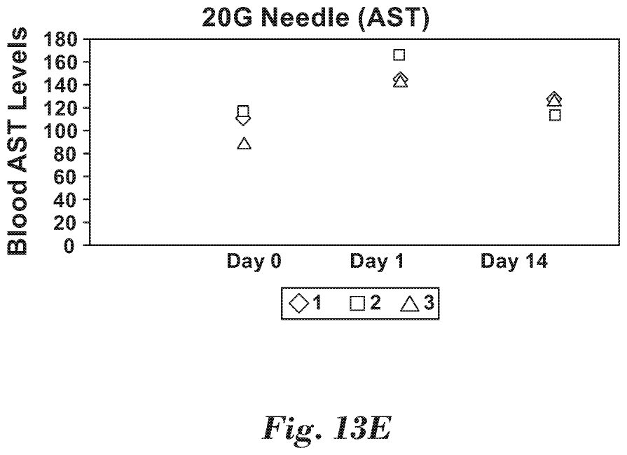

FIGS. 13A-13E demonstrate levels of representative liver enzymes (ALT, AST) at days 0, 1 and 14 post-treatment with Hovo2 particle (N=5) and 20 G needle (N=3); FIG. 13A demonstrates overall profile of liver enzymes; FIGS. 13B and 13C demonstrate individual profile for Hovo2 (ALT and AST, respectively); and FIGS. 13D and 13E demonstrate individual profile for 20 G needle group (where each differently colored circle represents individual animal, N of 5 for Hovo2 group and N of 3 for 20 G needle group).

Liver Injury Histology Data.

In general, livers collected from both the particle (Hovo2) and needle (20 G) treated animals at 1 hr, 3 hrs and 24 hrs post-procedure exhibited similar organ lesions including acute hepatic necrosis, hemorrhage, edema, neutrophilic inflammation and capsule damage. Interestingly, animals in the t=3 hrs and 24 hrs post treatment groups exhibited more pronounced histopathological markers of the liver damage vs. 1 hr cohort, as evidenced by hepatic necrosis, edema, inflammation, hemorrhage and capsule damage. Day 14 post-treatment animals consistently exhibited mild to no lesions compared to 1 hr, 3 hrs and 24 hrs groups. Pathological areas either were absent or limited to capsular or subcapsular areas.

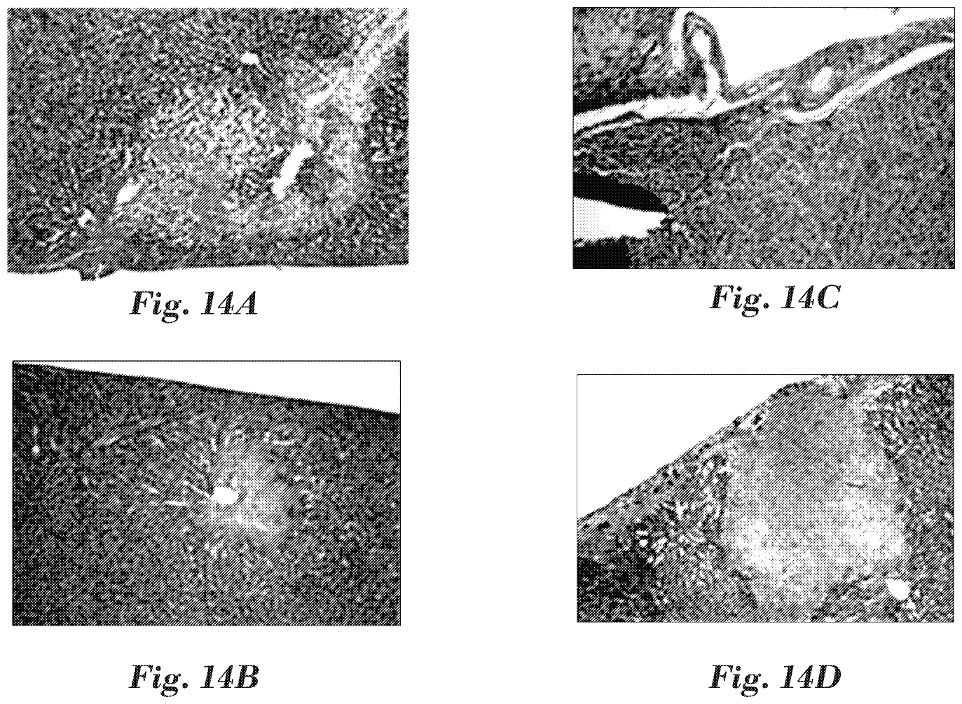

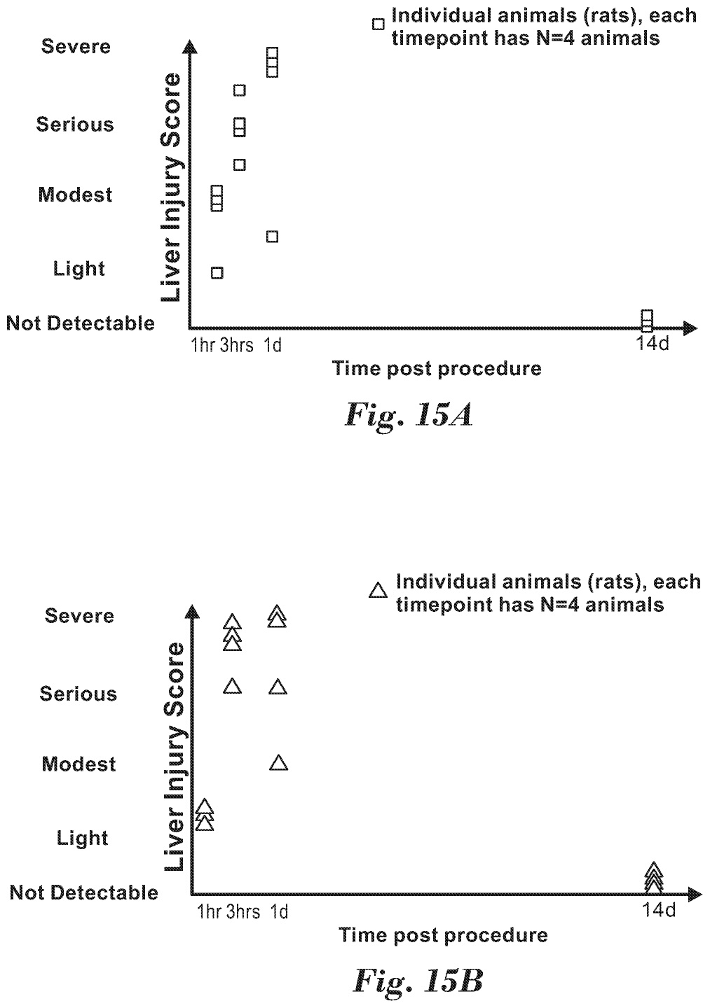

FIGS. 14A-14D show images of liver damage of rat treated with Hovo2 microbot taken at 1 hr, 3 hr, 24 hr, and 14 days respectively.

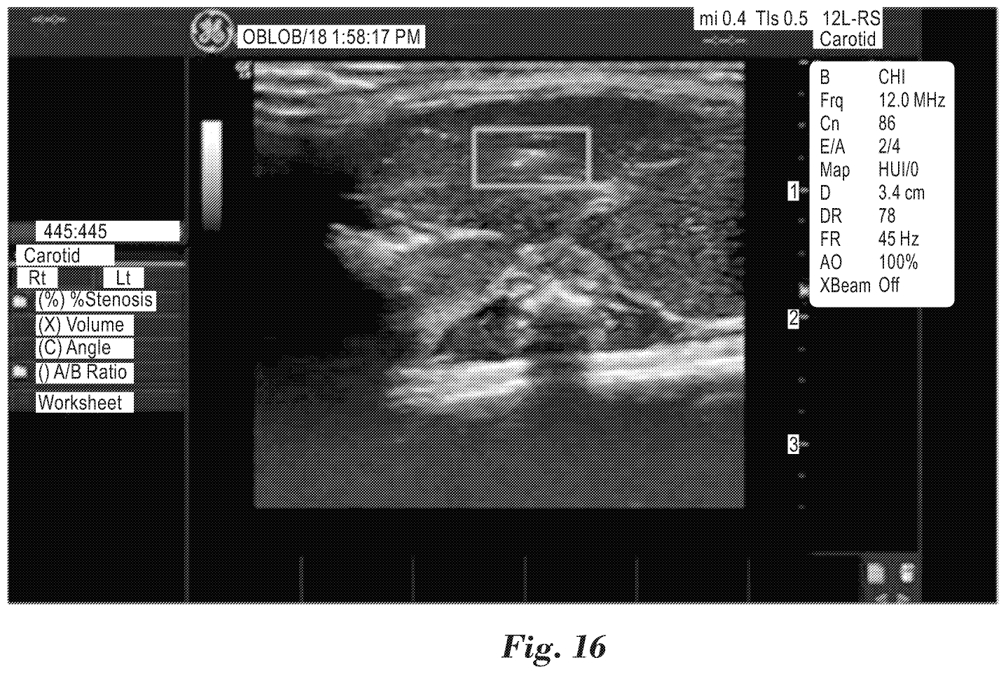

FIGS. 15A and 15B demonstrate liver injury score, observed in all sample's vs. time after treatment; FIG. 15A denotes injury score for animals treated with the Hovo2 microbot; FIG. 15B denotes injury score for animals treated with a 20 G needle.

A similar study in a small group of mice (BALBc, N=4) suggested that all treated animals recovered within 1 hr post procedure with either needle (G25) or particle (SKC8), similar to rats. No discomfort was observed or other treatment effects for the duration of the experiment (14 days). Limited liver damage analysis with particles suggested that the liver showed trend towards recovery by day 7 and recovered completely by day 14 of the studies.

Secondary Test Goals:

In order to meet the secondary goals defined above, we also validated the ultrasound-based visualization as potential imaging technique suitable to track the movement of the devices through tissue such as liver. The tracking software takes a frame by frame comparison of the ultrasound video pixel by pixel to track the microbot. The comparison is made using color schemes in Python software environment via OpenCV. If there is a large difference with subsequent frame with the previous one at certain pixels, past a predetermined motion threshold, the code recognizes this as the robot in motion. FIG. 16 demonstrates ultrasound image of spring based microbot, processed using image tracking software.

Furthermore, particles were reliably and reproducibly propelled ex vivo and in vivo for 5-12 mm distances in order to completely traverse a specific liver lobe. Table 6 below shows representative examples of in vivo evaluation of two particles vs. their traveling distance in through the liver in anesthetized rats.

TABLE 6

| | Rat | Distance | | Anatomical |
|---|---|---|---|---|
| Bot | (anesthetized) | Traveled | | Position |
| SKC8 | #1 | 12 | mm | Left Lateral |
| SKC8 | #2 | >5 | mm | Left Lateral |
| SKC8 | #3 | >5 | mm | Left Lateral |
| Hovo2 | #4 | 8 | mm | Left Lateral |
| Hovo2 | #5 | 8 | mm | Left Lateral |
| Hovo2 | #6 | 3 | mm | Left Lateral |
| Hovo2 | #7 | 6 | mm | Right Medial Lobe |

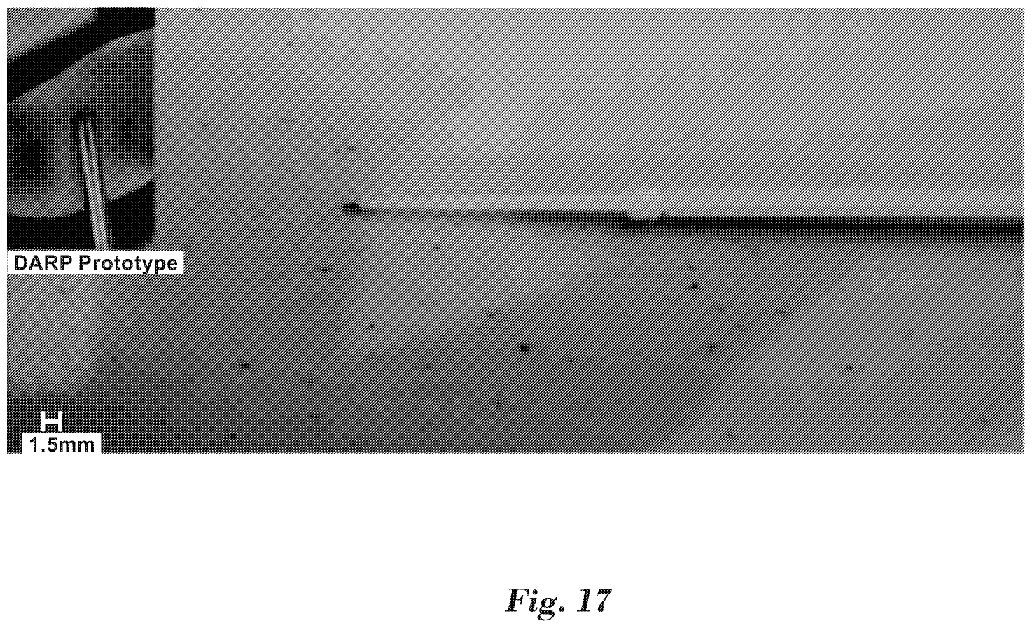

Also identified is a concept for the design and optimization of a magnetic particle retraction device suitable for safe, reliable and reproducible particles collection. The retraction device uses an Eppendorf tube with an ND52 0.8 mm magnet located on the tip as demonstrated in FIG. 17 including the microbot retraction prototype device in the top left corner. The device was used successfully for in vivo retrieval of the particles post treatment experiments.

Tests Summary:

Particles were successfully propelled through the liver in vivo using mouse and rat models and external magnetic stimuli. The histopathological assessment of tissue damage suggested that the liver sustained transient damage at both 3 and 24 hrs post-treatment. Key pathological observations included: hepatic necrosis, hemorrhage, edema, inflammation and capsule damage. Both particle and needle treated animals showed elevated liver enzymes at 24 hrs post treatment that correlated with the observed liver histopathology. In general, particle treated animals showed tendency for a comparable tissue damage and faster recovery as compared to the needle treated control group. All particles and needle-treated animals displayed complete recovery by day 14 post treatment as evidenced by both histology and representative blood biomarkers (ALT/AST). It was found that the propulsion of the currently provided particles through the liver tissue is safe and well tolerated in both mice and rats (N=40 total). No significant differences were detected in the livers of animals treated with the needle or the tested particle. Notably, all treated rodents (mice/rat) survived and behaved normally.

Using ultrasound, movement of the microbots through the liver was visualized and tracked and thereby the accuracy of propulsion was estimated. Moreover, a prototype device was designed, suitable for safe and accurate delivery & retraction of the particles and validated it both ex vivo and in vivo in murine models.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A propelling device, configured to propel through a medium, using external magnetic stimuli applied thereon, the device comprising:
a helical spring-like element; and
a cube, cuboid, prism, ellipsoid, disc-like, cylindrical magnet, accommodated within the helical element, wherein their longitudinal axes are aligned.

2. The device of claim 1, wherein the magnet is configured to respond to the applied magnetic stimuli and to rotate the helical element; and wherein the helical element is configured to convert rotary motion thereof into a translation motion along at least one of: the longitudinal axis, 2D trajectory, 3D trajectory; and thereby to propel the device through the medium.

3. The device of claim 1, wherein one of the following holds true:
the medium comprises at least one material selected from: viscoelastic medium, extracellular matrix, interstitial space, biological compartment, biological duct, biological vessel, biological node, biological tissue, biological organ;
the helical element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
at least one nickel-plated neodymium optionally selected from: N35, N38, N40, N42, N45, N48, N50, N52, and N55; or
at least one alternative permanent nano/micro magnet material selected from: samarium cobalt (SmCo), alnico, ceramic, ferrite.

4. The device of claim 1, wherein the front end of the helical element comprises a sharp and/or chiseled tip.

5. The device of claim 1, wherein the magnet is accommodated, at a front section, at a center section, or at a back section of the helical element.

6. The device of claim 1, wherein the magnet is encased with a layer of titanium vessel.

7. The device of claim 1, wherein at least part of the device is covered with—or embedded into a matrix that contains—an imaging agent, configured to facilitate visualization; the imaging agent optionally comprising at least one of: Rhodamine B, Fluorescein, microbubbles, microdefects, mesoporous silica nano- and micro-particles, and Upconversion Phosphors.

8. The device of claim 1, wherein the magnet is fixed to the helical element, optionally via an adhesive material comprising at least one of: epoxy, acrylics, polyurethane, UV curable, and cyanoacrylate based materials.

9. The device of claim 8, wherein the adhesive material is incorporated with mesoporous nano- or micro-silica particles, configured to enhance contrast under ultrasound radiation.

10. The device of claim 1, wherein:
the helical element comprises:
outer diameter ranging between 0.66-1.2 mm;
inner diameter ranging between 0.3-1.1 mm;
pitch length ranging between 0.5-2.2 mm;
length ranging between 1-5.6 mm;
the magnet comprises:
diameter ranging between 0.3-0.8 mm;
length ranging between 0.5-1.5 mm.

11. A propelling device, configured to propel through a medium, using external magnetic stimuli applied thereon, the device comprising:
a screw-like element, characterized by conical- or cylindrical-core and a helical ridge;
a cylindrical magnet, accommodated within a hole drilled in the cylindrical core, wherein their longitudinal axes are aligned.

12. The device of claim 11, wherein the magnet is accommodated at a front section or a back section of the cylindrical core.

13. The device of claim 11, wherein the magnet is configured to respond to the applied magnetic stimuli and to rotate the helical element; and wherein the screw-like element is configured to convert rotary motion thereof into translation motion along at least one of: the longitudinal axis, 2D trajectory, 3D trajectory; and thereby to propel the device through the medium.

14. The device of claim 11, wherein one of the following holds true:
the medium comprises at least one material selected from: viscoelastic medium, extracellular matrix, interstitial space, biological compartment, biological duct, biological vessel, biological node, biological tissue, biological organ;
the screw-like element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
at least one nickel-plated neodymium optionally selected from: N35 N38, N40, N42, N45, N48, N50, N52, and N55; or
at least one alternative permanent nano/micro magnet material selected from: samarium cobalt (SmCo), alnico, ceramic, ferrite.

15. The device of claim 11, wherein:
the screw-like element comprises:
length of ranging between 1.1-1.7 mm;
outer diameter ranging between 0.57-0.65 mm;
inner diameter ranging between 0.38-0.5 mm;
pitch ranging between 0.34-0.60 mm;
the hole diameter ranging between 0.2-0.4 mm;
the magnet comprises:
diameter ranging between 0.2-0.5 mm;
length ranging between 0.5-1.5 mm.

16. A propelling device, configured to propel through a medium, using external magnetic stimuli applied thereon, the device comprising:
a propelling element comprising:
a drill-bit-like element or a chisel-like, configured to vacate the surrounding medium as it rotates through; or
a screw-like element, characterized by a cylindrical core and a helical ridge; or
a twisted-ribbon-like element;
a cylindrical magnet, attached to the back end of the propelling element, wherein their longitudinal axes are aligned.

17. The device of claim 16, wherein the diameter of the cylindrical magnet equals to—or smaller then—the outer diameter of the propelling element.

18. The device of claim 16, wherein the magnet is attached to the back end of the propelling element via an adhesive material, optimally comprising at least one of: epoxy, acrylics, polyurethane, UV curable, and cyanoacrylate based materials.

19. The device of claim 16, wherein the magnet is configured to respond to the applied magnetic stimuli and to rotate the propelling element; and wherein the propelling element is configured to convert rotary motion thereof into translation motion along at least one of: the longitudinal axis, 2D trajectory, 3D trajectory; and thereby to propel the device through the medium.

20. The device of claim 16, wherein one of the following holds true:
the medium comprises at least one material selected from: viscoelastic medium, extracellular matrix, interstitial space, biological compartment, biological duct, biological vessel, biological node, biological tissue, biological organ;
the propelling element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
at least one nickel-plated neodymium optionally selected from: N35, N38, N40, N42, N45, N48, N50, N52, and N55; or
at least one alternative permanent nano/micro magnet material selected from: samarium cobalt (SmCo), alnico, ceramic, ferrite.

21. The device of claim 16, wherein:
the device comprises:
length of ranging between 1.0-3.3 mm;
propelling element's outer diameter ranging between 0.5-1.5 mm;
propelling element's inner diameter ranging between 0.2-0.85 mm;
propelling element's pitch ranging between 0.44-0.81 mm;
the magnet comprises:
diameter ranging between 0.2-0.6 mm;
length ranging between 0.5-1.5 mm.

22. A propelling device, configured to propel through a medium, using external magnetic stimuli applied thereon, the device comprising:
a tube, characterized by a carved helical-like front section;
a cylindrical magnet, accommodated within the bore of the tube, at its back section, wherein their longitudinal axes are aligned.

23. The device of claim 22, wherein the magnet is configured to respond to the applied magnetic stimuli and to rotate the tube; and wherein the tube's carved helical-like front section is configured to convert rotary motion thereof into translation motion along at least one of: the longitudinal axis, 2D trajectory, 3D trajectory; and thereby to propel the device through the medium.

24. The device of claim 22, wherein one of the following holds true:
the medium comprises at least one material selected from: viscoelastic medium, extracellular matrix, interstitial space, biological compartment, biological duct, biological vessel, biological node, biological tissue, biological organ;
the tube comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
at least one nickel-plated neodymium optionally selected from: N35, N38, N40, N42, N45, N48, N50, N52, and N55; or
at least one alternative permanent nano/micro magnet material selected from: samarium cobalt (SmCo), alnico, ceramic, ferrite.

25. The device of claim 22, wherein:
the tube comprises:
length of ranging between 1.7-3.5 mm;
outer diameter ranging between 0.76-0.83 mm;
inner diameter ranging between 0.3-0.6 mm;
pitch of the helical section ranging between 0.51-1.50 mm;
the magnet comprises:
diameter ranging between 0.3-0.6 mm;
length ranging between 0.5-3.0 mm.

26. A propelling device, configured to propel through a medium, using external magnetic stimuli applied thereon, the device comprising:
a wedge-like element, configured to pierce through the medium as it translates through; and
a magnet, attached to the back end of the wedge-like element, wherein the magnet's longitudinal axis is parallel to the wedge-like element's back end wall.

27. The device of claim 26, wherein the magnet is attached to the back end of the wedge-like-element via an adhesive material, optimally comprising at least one of: epoxy, acrylics, polyurethane, UV curable, and cyanoacrylate based materials.

28. The device of claim 26, wherein the magnet is configured to respond to the applied magnetic stimuli and to translate the wedge-like-element, and thereby to propel the device through the medium.

29. The device of claim 26, wherein one of the following holds true:
the medium comprises at least one material selected from: viscoelastic medium, extracellular matrix, interstitial space, biological compartment, biological duct, biological vessel, biological node, biological tissue, biological organ;
the wedge-like element comprises at least one material having Young's modulus stiffness above 1 GPa, optionally selected from: Polypropylene, Polystyrene, high impact Polystyrene, Acrylonitrile butadiene styrene, Polyethylene terephthalate, Polyester, Polyamides (Nylons), Poly (vinyl chloride) (PVC), glass, ceramics, metals selected from: copper, bronze titanium, titanium related alloys, stainless steel, gold;
the magnet comprises:
at least one nickel-plated neodymium optionally selected from: N35, N38, N40, N42, N45, N48, N50, N52, and N55; or
at least one alternative permanent nano/micro magnet material selected from: samarium cobalt (SmCo), alnico, ceramic, and ferrite.

30. The device of claim 26, wherein:
the wide-like-element comprises:
side length ranging between 0.2-2.5 mm;
height ranging between 0.2-5.0 mm;
head angle ranging between 25-75 deg;
the magnet comprises:
diameter ranging between 0.2-0.6 mm;
length ranging between 0.2-3.0 mm.
